(19) United States
(12) Patent Application Publication
(10) Pub. No.: US 2021/0012879 A1
(43) Pub. Date: Jan. 14, 2021

(54) METHOD FOR DETERMINING FIBER INTAKE

(71) Applicant: CARBIOTIX AB, VIKEN (SE)

(72) Inventor: Peter FALCK, KÄVLINGE (SE)

(21) Appl. No.: 16/762,489

(22) PCT Filed: Nov. 9, 2018
(86) PCT No.: PCT/SE2018/051145

§ 371 Date: May 7, 2020

(30) Foreign Application Priority Data
Nov. 9, 2017 (SE) 1700275-9

(51) Int. Cl. G16H 20/60; G16H 10/40

(52) U.S. Cl. CPC G16H 20/60; G16H 10/40

(57) ABSTRACT

The present invention relates to a method for controlling fiber intake in mammals, comprising the steps: a) providing at least one input data; b) based on said input data, generating at least one output data; wherein the input data is based on information selected from the group consisting of host DNA, host metabolism, host colonic microbial composition, gene activity, metabolic activity, host specific parameters, and any combinations thereof wherein the output data is selected from the group consisting of information on fiber consumption, fiber supplementation and, any combinations thereof. The present invention further relates to a system and use of said method.

### FIELD OF THE INVENTION

The present invention relates to how fiber intake can be determined based on a set of different inputs. Further the invention also describes how the input and output is communicated through a control unit.

### TECHNICAL BACKGROUND

People today are consuming to little fiber in their diet. It is well recognized that there is a fiber gap which in turn increase the risk of many chronic diseases such as cardiovascular disease, diabetes, obesity, and cancer. It is difficult to address this issue due to a low content of fiber, especially soluble fiber, in the western diet and it is therefore important to supplement the diet with fiber rich foods or supplements. Considerable proof of the benefits of soluble fiber are now available. They are fermented by the fecal microbiota producing health promoting short chain fatty acids acetate, propionate, and butyrate. Certain groups of bacteria are especially important and benefits from a diet rich in soluble fiber. Bifidobacteria is one of them and is considered as one of the most important groups of beneficial bacteria due their ability to stimulate immune system development, produce vitamins, inhibit pathogens, reduce ammonia and cholesterol in the blood and help to restore a healthy gut after antibiotic treatment. Another group of important bacteria are the butyrate producing bacteria belonging to clostridial clusters IV and XIVa. They produce butyric acid through breakdown of carbohydrates or through cross-feeding with e.g. bifidobacteria. Butyrate is well recognized as an important anti-inflammatory substance which helps to maintain a healthy gut barrier function. Many different factors impact the fiber requirement for an individual most importantly age, gender, and the bacterial composition in the gastro intestinal tract.

### SUMMARY OF THE INVENTION

The current invention describes how to determine fiber intake in a mammal such as humans or dogs and cats (FIG. 1).

In one aspect the present invention provides a method for controlling fiber intake in mammals, comprising the steps:

a) providing at least one input data;

b) based on said input data, generating at least one output data; wherein the input data is based on information selected from the group consisting of host DNA, host metabolism, host colonic microbial composition, gene activity, metabolic activity, host specific parameters, and any combinations thereof wherein the output data is selected from the group consisting of information on fiber consumption, fiber supplementation and, any combinations thereof. The input data may be obtained using at least one sample measurement device to measure a sample obtained from a mammal to provide information from said information group as basis for the input data.

In one embodiment, the present invention provides input data further comprising input measurements, wherein the input measurements comprise physiological reported measurements such as blood pressure, triglycerides, cholesterol, white blood cell count, vitamin count, immunoglobulins, breath hydrogen, and/or breath methane. In one embodiment input data are from measurements in samples earlier obtained. The input data may be obtained using input measurements using at least one sample measurement device to measure a sample obtained from a mammal to provide information from said information group as basis for the input data.

In one embodiment, the input data comprise information on age, gender, body mass index, physical activity, stress, sleep, disease state, diet status, gut status, life style habits and/or antibiotic use.

In one embodiment, the output data comprise information on fiber intake preferably wherein the fiber is present in foods such as cereals, vegetables, legumes, berries, and fruits such as bananas, pears, and apples. It is clear that the foods are not limited to the foods specified in the claim and can be of any kind or processed in many different ways.

In one embodiment, the output data comprise information on a fiber supplement based on fibers containing non- or partially digestible polysaccharides and/or oligosaccharides comprising modified or unmodified starch and partial hydrolysates thereof, inulin or partially hydrolyzed inulin, natural oligofructoses, fructo-oligosaccharides (FOS), lactulose, lactosucrose, soybean-oligosaccharides (SOS), galactomannan and suitable partial hydrolysates thereof, manno-oligosaccharides (MOS), indigestible polydextrose, acemannan, various gums and pectin and partial hydrolysates thereof, indigestible dextrin and partial hydrolysates thereof, trans-galacto-oligosaccharides (GOS), xylo-oligosaccharides (XOS), xylan, arabinoxylan, arabinogalactan, arabino-xylooligosaccharides (AXOS), beta-glucan and partial hydrolysates thereof, chito-oligosaccharides (COS), glucomano-oligosaccharides (GMOS), arabinooligosaccharides (AOS), pectin-oligosaccharides (POS), laminar-oligosaccharides, human milk oligosaccharides (HMO), bovine milk oligosaccharides (BOS). It is clear that the non-digestible or partially digestible fibers mentioned above is not limit to the examples given and can be of any origin or processed in a number of different ways. In one embodiment the present invention comprises a mixture of at least two of the fibers disclosed.

In one embodiment, the output data comprise information on a fiber amount intake, preferably a daily intake of from 0.01 to 100 g per day, preferably from 0.1 to 50 g per day, preferably from 0.5 to 20 g per day, preferably from 0.5 to 15 g per day, preferably from 0.5 to 10 g per day.

In one embodiment, the output data comprise information on frequency of consumption of fiber, wherein the frequency is at a minimum once a month, preferably once a week, preferably at least once and/or twice every day, up to 4 times a day.

In one embodiment, the output data comprise information on at least one other substance besides information on fiber intake and/or supplement, preferably the at least one other substance is selected from the group consisting of strains of microorganisms, vitamins, minerals, antioxidants, fatty acids, plant and/or herbal extracts, and any combinations thereof.

In one embodiment the method of the present invention further comprises the steps of:
c) transferring the input data into a control unit comprising a database, a file, a data processor for receiving input data and converting the received data to an output data; and
d) displaying the output data of step c) for a user connected to a network. In one embodiment the method of the present invention further comprises the steps of:
e) receiving the input data by a control unit;
f) converting the received input data of step c) to at least one output data;
g) upholding at least a predetermined set point for the output data by controlling fiber intake.

In one embodiment the method of the present invention further comprises the step:
h) presenting output data to control fiber intake together with data showing microbial composition and/or gene activity and/or metabolic activity.

In one embodiment the method of the present invention further comprises the steps of:
c) transferring the input data into a control unit comprising a database, a file, a data processor for receiving input data and converting the received data to an output data; and
d) displaying the output data of step c) for a user connected to a network.
e) receiving the input data by a control unit;
f) converting the received input data of step c) to at least one output data;
g) upholding at least a predetermined set point for the output data by controlling fiber intake.
h) presenting output data to control fiber intake together with data showing microbial composition and/or gene activity and/or metabolic activity.

In one embodiment, the control unit is connected to a remote control unit via a network, the remote control unit having user interface capability, so that input data may be transferred from the control unit and output data may be generated and displayed for a user being connected to the remote control unit via the network.

In one embodiment, the remote control unit is located in a cloud-based computing environment.

In one embodiment, the remote control unit is a virtual control unit.

In one embodiment, the remote control unit have a database, a file, a data processor for receiving input data and converting the input data to output data and displaying for a user connected to the network.

In one embodiment, the control unit having user interface capability, so that input data and output data may be displayed for a user being connected to the control unit via the network.

In one embodiment, the control unit is connected to a remote control unit, having user interface capability, via a network, so that input data may be transferred from the control unit and displayed for a user being connected to the remote control unit via the network.

In one embodiment, the output is generated with an algorithm, machine learning or artificial intelligence.

In one embodiment, the present invention is a method for monitoring and/or controlling fiber intake.

In one aspect the present invention provides a system for controlling fiber intake in mammals comprising a control unit adapted to receive input data and provide output data, at least one sample measurement device adapted to measure a sample obtained from a mammal to provide input data for the control unit, and optionally a remote control unit connected to said control unit via a network.

### SHORT DESCRIPTION OF THE DRAWINGS

FIG. 1 discloses a drawing showing the flow of input into a control unit to determine an output on fiber intake.

FIG. 2 discloses a user interface for the present invention, wherein a) shows a graph over time with increasing fiber dosage and effect on Bifidobacteria of the gut of a user, which provide recommendation to increase fiber intake by food or supplement based on percentage of bifidobacteria, b) shows a graph over time with increasing fiber dosage and effect on Bifidobacteria of the gut of a user, which provide recommendation to keep the same amount of fiber intake by food or supplement based on percentage of bifidobacteria in view of reached target of the last fiber amount used.

FIG. 3 discloses a schematic block diagram of a system for controlling fiber intake in mammals according to the present invention.

### DETAILED DESCRIPTION OF THE INVENTION

The wording “fiber” and “fiber intake” as used herein is intended to be interpreted as fibers used in the present invention, and which are used as ingoing components. The fibers referred to herein are preferably soluble fibers. The wording fiber is primarily defined as a soluble fiber. The soluble fiber may be selected from oligosaccharide(s) and/or polysaccharide(s), which may be fully or partially fermented by the gut microbiota. When fiber used as fiber intake is present in foods, the recommendation of food items is primarily based on the soluble components of the total fiber content. Thus, the fiber intake recommendation may be based on the soluble fibers of food item(s).

The present way of determining fiber intake may be made using a system 1 for controlling fiber intake in a mammal comprising a control unit 4. The system may further comprise at least one sample measurement device 3 adapted to measure parameters of a sample 2 obtained from the mammal. The mammal sample 2 may be a base to provide the input data and/or output data. The system may comprise at least one first sample measurement device providing input data and/or at least one second sample measurement device providing results for output data, or output data itself.

The fiber intake may be adapted to be controlled to uphold at least a predetermined set point value in relation to the value(s) measured by said sample measurement device(s).

The control unit 4 of the present system may have a database, a file, and a data processor for receiving input data and converting the received input data to output data and displaying for a user connected to the network. The input data and/or output data may be correlated with at least a predetermined set point value of the fiber intake. The control unit 4 may have user interface capability, so that input data and/or output data may be displayed for a user being connected to the control unit via the network.

The control unit 4 may be connected to a remote control unit 5 via a network, the remote control unit having user interface capability, so that input data and/or output data may be transferred from the control unit to be displayed for a user being connected to the remote control unit via the network. The remote control unit 5 may be located in a cloud-based computing environment. The remote control unit 5 may be a virtual control unit. The remote control unit 5 may have a database, a file, and a data processor for receiving data and converting the received input data to output data and displaying for a user connected to the network. The input data and/or output data may be correlated with at least a predetermined set point value of the fiber intake.

In order to address the problem of determining the fiber intake for an individual, a measurement of bacteria present in the microbiota was performed. In the first example a test group of 6 people took a fiber supplement comprising of 3 g arabinogalactan every day for a total period of 3 weeks. The test subjects sent in a fecal swab sample before and after 3 weeks of consuming arabinogalactan. Bacterial DNA was extracted and QPCR was used to measure the level of bifidobacteria present in each sample. All results were stored together with each user's profile in a database. Based on the results an algorithm was used to determine if the test subject needed a higher or lower dose of fiber to find the optimal dose to fill each individual's fiber gap. The algorithm comprises a simple decision (if bifidobacteria count <3% increase fiber dose or if bifidobacteria count >5% decrease fiber dose, otherwise keep same fiber dose). The output data on fiber intake was presented to each user connected to a network through a web browser. Together with the presented recommendations on fiber intake where also the percentage of bifidobacteria.

TABLE 1
Percentage of bifidobacteria and recommendation
on fiber dose for each test subject after a 3-week
trial on 3 g of arabinogalactan per day

| | |
|---|---|
| Bifidobacteria Count | Recommendation |

| Age | BMI | Week 0 | Week 3 | on fiber dose |
|---|---|---|---|---|
| 78 | 22 | 0.6% | 3.5% | Keep dose |
| 46 | 23 | 2.0% | 2.8% | Increase |
| 58 | 25 | 5.9% | 8.4% | Decrease |
| 49 | 27 | 1.7% | 3.2% | Keep dose |
| 33 | 21 | 0.1% | 9.1% | Decrease |
| 49 | 30 | 0.5% | 1.4% | Increase |

The fiber intake for an individual may be determined by increasing the amount of soluble fiber until reaching a predetermined value, e.g. a specific amount of bacteria present in the microbiota. Bifidobacteria may be used as an indicator in the microbiota. For example, the fiber intake for an individual may be determined by increasing the amount of soluble fiber until reaching 0.1-50% bifidobacteria present in the microbiota, such as 1%-25%, 2%-25%, 5%-25%, or 5%-15% bifidobacteria present in the microbiota. The fiber intake for an individual may be determined by increasing the amount of soluble fiber until reaching at least 2% bifidobacteria, such as at least 5% bifidobacteria, present in the microbiota, e.g. as described in following Example 2. The fiber intake may be a daily intake of about 0.01-100 g/day, such as 0.1-50 g/day, 0.5-20 g/day, 0.5-15 g/day, 0.5-10 g/day. Said amount of daily fiber intake may be based on soluble fiber, i.e. based on the soluble components of the total fiber content. 0.2-50 g/day, such as 0.5-40 g/day, 1-30 g/day, or 5-20 g/day. The fiber intake may be increased with 0.5 g/day for each subsequent predetermined time period, such as every month, until bifidobacteria count reach said at least 5%. This means that for each new time period, such as a month, the dosage of that time period (e.g. month) may be increased with 0.5 g/day compared to the daily dosage of the previous time period (e.g. month). The same fixed dosage is normally used within a specific time period, such as a specific month of fiber intake. The initial starting point of the fiber intake may be individualized, and may be based e.g. on gender, age, and/or BMI. Starting point may be selected different between males and females, e.g. women may start with a lower fiber dosage than men. Women may start with a fiber intake of 1-5 g/day, such as 2-3 g/day, or about 2 g/day, and men may start with 2-6 g/day, such as 2-5 g/day, or about 3 g/day. Some subjects may not reach the aimed target of at least 5% bifidobacteria present in the microbiota. For subjects which are not reaching 5% bifidobacteria with a 5 g/day dose, the dose may be increased, e.g. to first about 10 g/day, and then if needed to about 20 g/day.

The amount of soluble fiber required to reach at least 2% bifidobacteria, such as 5% bifidobacteria, may vary considerably between subjects. Different amount of fiber may be needed in order to have the same impact on gut health for different people.

Having the feedback of gut microbiota composition when recommending fiber intake clearly have a very different technical effect on the recommendation compared with recommendations solely relying on Recommended Daily Intake (RDI) or input such as gender, age and BMI. The study demonstrates that if people have different gut microbiota compositions they will react very different to the same amount of fiber. People having an initial low number of bifidobacteria require more fiber before a measurable increase in bifidobacteria can be detected. However, people already having a greater population of bifidobacteria generally react faster to an increase in fiber.

Furthermore, by having a continuous intermittent sampling, e.g. of fecal matter, it possible to adjust the recommendation on a regular basis. By obtaining a monthly sample of the gut microbiota composition, e.g. from fecal samples, it may be possible to adjust the recommendation on a regular, such as a monthly, basis. Regular testing to adjust the recommended dose of fiber ensure that the user benefits from getting the right amount of fiber through their diet or supplement every day. Without this feedback it is not possible to know if the subject will benefit from a general advice on fiber intake because everyone reacts different to fiber. Recommendations of fiber intake is therefore not always good advice for improving gut health unless a proper feedback mechanism is used to adjust the advice for each individual. Current methods of recommending fiber does not utilize a feedback loop from the microbiota and can therefore underestimate the need of fiber for certain individuals leaving them without the proper amount of fiber in their diet and on the long term increasing their risk of disease.

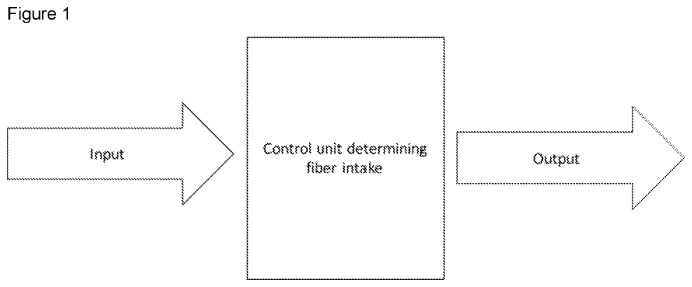

FIG. 1 show the flow of input into a control unit to determine an output on fiber intake.

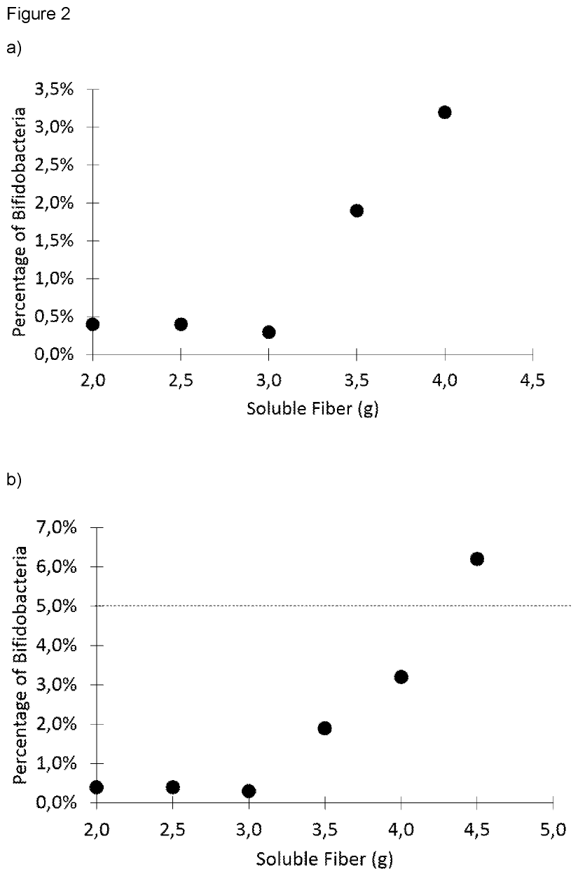

FIG. 2 discloses information that may be presented to a user via a user interface. The graph a) shows test data obtained from the user over time. The user has increased the dosage of soluble fiber and now reached a fiber intake of 4.0 g.

The current result of the percentage of Bifidobacteria is then analyzed to 3.2% based on a sample provided from the user. The user interface may further provide a recommendation to increase fiber intake by food or supplement based on percentage of bifidobacteria. The subsequent time period, e.g. a month, the recommended fiber intake amount may be increased to 4.5 g of soluble fiber. The graph b) shows test data obtained from the user after an additional time period. The user has increased the dosage of soluble fiber and now reached a fiber intake of 4.5 g. The current result of the percentage of Bifidobacteria is then analyzed to 6.2% based on a sample provided from the user. The user interface may further provide a recommendation to increase or maintain fiber intake by food or supplement based on percentage of bifidobacteria. The subsequent time period, e.g. a month, the recommended fiber intake amount may be maintained at 4.5 g of soluble fiber as the result of the bifidobacteria has reached a predetermined set value, here of about 5.0% bifidobacteria. The user interface may apart from or instead of a disclosure of fiber intake dosage in relation to gut indicators, such as Bifidobacteria, and the recommended intake of soluble fibers, further provide input on examples of food items recommended to the user which are intended to cover the recommended intake of soluble fibers. If a user is to consume 4.5 g of soluble fiber based on the latest tests of the user, e.g. blood sample or gut health samples via fecal sample, a recommendation of food items of choice containing the desired fiber content may be provided in accordance with table 2.

TABLE 2
Input to a user regarding alternative
food intake instead of supplement

| Name | Portion | Amount |
|---|---|---|
| Apple | Medium size | 1 |
| Carrots, raw | 120 ml | 3 |
| Figs raw | 1 large | 1 |
| Balance: +4.5 g | Calories: 205 kcal | |

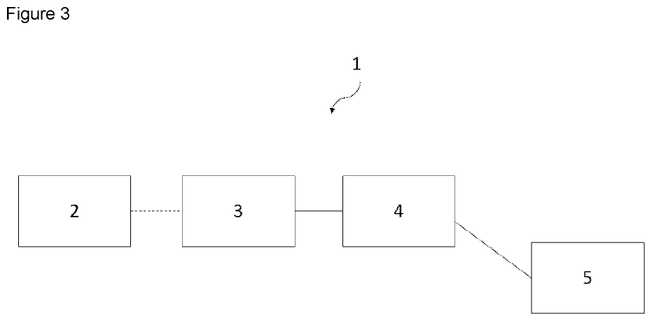

FIG. 3 discloses an embodiment of the system for controlling fiber intake in mammals according to the present invention. The system 1 for controlling fiber intake in mammals comprises a control unit 4, at least one sample measurement device 3 adapted to measure parameters of a sample 2 obtained from a mammal. The control unit 4 may be connected to a remote control unit 5 via a network.

From the description above follows that, although various embodiments of the invention have been described and shown, the invention is not restricted thereto, but may also be embodied in other ways within the scope of the subject-matter defined in the following claims.

### EXAMPLES

### Example 1

### Determining Fiber Intake in Test Subjects

Human fecal swab samples were collected from a test group of adult men and women. The fecal samples were bead beaten in a lysis buffer for 20 minutes. Bacterial DNA was isolated with magnetic beads and eluted in RNase free water. Total DNA was quantified using 260 nm using a nano-drop spectrophotometer. Quantitative PCR amplification and detection were carried out using primers for bifidobacteria primer pair 5′-3′ (ACTCCTACGGGAGGCAGCAGT & ATTACCGCGGCTGCTGGC and total bacteria primer pair 5′-3′ (CTCCTGGAAACGGGTGGT & GCTGCCTCCCGTAGGAGT). PCR amplification and detection was performed using an Quantstudio 3 (Applied Biosystems, Darmstadt, Germany) in optical-grade 96-well plates sealed with optical sealing tape. Each reaction mixture (20 μl) was composed of 10 μl of SYBR Green PCR Master Mix (Applied Biosystems, Darmstadt, Germany), 2 μl primer mix (10 pmol/μl each), 9 μl sterile distilled H2O, and 1.5 μl stool DNA (10 ng/μl). For the negative control, 2 pl of sterile distilled H2O was added to the reaction solution instead of the template DNA solution. A melting curve analysis was carried out following amplification to distinguish the targeted PCR product from the nontargeted PCR product. Each real-time PCRs were performed in triplicate, and average values were used for calculations. PCR conditions comprise one cycle of 50° C. for 2 min, 95 ° C. for 2 min and then 40 cycles of 95 ° C. for 30 s, 60° C. for 30 s, and 72° C. for 60 s. The fraction of bifidobacteria was calculated as 1/2̂(delta Ct)), where delta Ct is the difference between the cycles for total bacteria and bifidobacteria.

Based on the fraction of bifidobacteria an algorithm decided if the fiber dose should be reduced or increased. If bifidobacteria >5% decrease fiber dose, if bifidobacteria is <5% and >3% keep the same fiber dose, if bifidobacteria <3% increase fiber dose.

### Example 2

### Determining Fiber Intake by Increasing Amount of Soluble Fiber

Subjects where enrolled with an BMI<25 and between 40-50 years of age, both men and women. Fecal swabs were collected and analyzed for bifidobacteria as described in Example 1. However, the samples were collected every month instead of every week and the amount of soluble fiber arabinogalactan started at 2.0 g/day for women and 3.0 g/day for men, respectively. The fiber was increased with 0.5 g/day every month until the bifidobacteria reached more than 5% of total bacteria. The required fiber varied considerably between different people as can be seen in Tables 3 and 4.

TABLE 3
Amount of soluble fiber required to reach an
amount of bifidobacteria for different men

| Arabinogalactan | RDI | Percentage of Bifidobacteria | | | | |
|---|---|---|---|---|---|---|
| (soluble fiber) | (men) | Man 1 | Man 2 | Man 3 | Man 4 | Man 5 |
| g | % | % | % | % | % | % |
| 2.0 | 21 | | | | | |
| 2.5 | 26 | | | | | |
| 3.0 | 32 | 0.3 | 2.0 | 0.2 | 1.0 | 1.3 |
| 3.5 | 37 | 0.1 | 2.2 | 0.3 | 3.0 | 2.0 |
| 4.0 | 42 | 0.1 | 2.0 | 0.1 | 3.7 | 2.0 |
| 4.5 | 47 | 0.1 | 4.8 | 0.6 | 5.0 | 1.9 |
| 5.0 | 53 | 0.9 | 7.8 | 0.9 | | 6.5 |
| 10.0 | 105 | 1.8 | | 1.2 | | |
| 20.0 | 211 | 5.4 | | 6.7 | | |

TABLE 4
Amount of soluble fiber required to reach an amount
of bifidobacteria for different women

| Arabinogalactan | RDI | Percentage of Bifidobacteria | | | | |
|---|---|---|---|---|---|---|
| (soluble fiber) | (women) | Woman 1 | Woman 2 | Woman 3 | Woman 4 | Woman 5 |
| g | % | % | % | % | % | % |
| 2.0 | 32 | 2.0 | 0.4 | 0.5 | 4.0 | 0.0 |
| 2.5 | 40 | 2.3 | 0.4 | 0.3 | 4.2 | 0.0 |
| 3.0 | 48 | 4.9 | 0.3 | 1.4 | 5.4 | 0.4 |
| 3.5 | 56 | 12.4 | 1.9 | 4.0 | | 0.6 |
| 4.0 | 64 | | 3.2 | 5.1 | | 1.3 |
| 4.5 | 72 | | 6.2 | | | 3.2 |
| 5.0 | 80 | | | | | 5.2 |
| 10.0 | 160 | | | | | |
| 20.0 | 320 | | | | | |

The result on percentage of bifidobacteria for each participant was stored in a database and was used as input data in an algorithm to determine if the fiber amount should be increased or not for each individual. The output from the algorithm was the fiber amount that was recommended for the following month. This was repeated in a loop until the desired amount of 5% bifidobacteria was reached for each participant. The data was displayed to the users when connected to a network through a user interface as exemplified in FIG. 2.

As can be seen in Tables 3 and 4, two subjects required 20 g/day of soluble fiber (equal to approximately 200% of RDI for fiber) while the other subjects only required 4.5-5 g/day of soluble fiber (equal to 47-53% of RDI for fiber) to reach at least 5% bifidobacteria. This clearly demonstrates that different amount of fiber is needed in order to have the same impact on gut health for different people. Further, this demonstrates the need for gut composition analysis as an input for accurately recommending fiber intake to improve health.

1. A method for controlling fiber intake in mammals, comprising the steps:
a) providing at least one input data; and
b) based on said input data, generating at least one output data;
wherein the input data is based on information selected from the group consisting of host DNA, host metabolism, host colonic microbial composition and/or gene activity and/or metabolic activity, host specific parameters and, any combinations thereof and wherein the output data is selected from the group consisting of information on fiber consumption, fiber supplementation and, any combinations thereof.

2. Method according to claim 1, wherein the input data further comprise input measurements, wherein the input measurements comprise physiological reported measurements such as blood pressure, triglycerides, cholesterol, white blood cell count, vitamin count, immunoglobulins, breath hydrogen, and/or breath methane, optionally wherein the input data further comprise information on age, gender, body mass index, physical activity, stress, sleep, disease state, diet status, gut status, life style habits and/or antibiotic use.

3. (canceled)

4. Method according to claim 1, wherein the output data comprise information on fiber intake preferably wherein the fiber is present in foods such as cereals, vegetables, legumes, berries, and fruits such as bananas, pears, and apples.

5. Method according to claim 1, where in the output data comprise information on a fiber supplement based on at least one fiber selected from the group consisting of fibers containing non- or partially digestible polysaccharides, oligosaccharides comprising modified or unmodified starch, and partial hydrolysates thereof, inulin and partially hydrolyzed inulin, natural oligofructoses, fructo-oligosaccharides (FOS), lactulose, lactosucrose, soya bean-oligosaccharides (SOS), galactomannan and suitable partial hydrolysates thereof, manno-oligosaccharides (MOS), indigestible polydextrose, acemannan, various gums and pectin and partial hydrolysates thereof, indigestible dextrin and partial hydrolysates thereof, trans-galacto-oligosaccharides (GOS), xylo-oligosaccharides (XOS), xylan, arabinoxylan, arabinogalactan, arabino-xylooligosaccharides (AXOS), beta-glucan and/or partial hydrolysates thereof, chito-oligosaccharides (COS), glucomano-oligosaccharides (GMOS), arabinooligosaccharides (AOS), pectin-oligosaccharides (POS), laminar-oligosaccharides, human milk oligosaccharides (HMO), bovine milk oligosaccharides (BOS), and any combinations thereof.

6. Method according to claim 1, where in the output data comprise information on a fiber amount intake, preferably a daily intake of from 0.01 to 100 g per day, preferably from 0.1 to 50 g per day, preferably from 0.5 to 20 g per day, preferably from 0.5 to 15 g per day, preferably from 0.5 to 10 g per day.

7. Method according to claim 1, wherein the output data comprise information on frequency of consumption of fiber, wherein the frequency is at a minimum once a month, preferably once a week, preferably at least once and/or twice every day, up to 4 times a day.

8. Method according to claim 1, wherein output data comprise information on at least one other substance besides information on fiber intake and/or supplement, preferably the at least one other substance is selected from the group consisting of strains of microorganisms, vitamins, minerals, antioxidants, fatty acids, plant and/or herbal extracts, and any combinations thereof.

9. Method according to claim 1, further comprising the steps of:
c) transferring the input data into a control unit comprising a database, a file, a data processor for receiving input data and converting the received data to an output data;
d) displaying the output data of step c) for a user connected to a network;
e) optionally receiving the input data by a control unit;
f) optionally converting the received input data of step c) to at least one output data; and
g) optionally upholding at least a predetermined set point for the output data by controlling fiber intake; and
h) optionally presenting output data to control fiber intake together with data showing microbial composition and/or gene activity and/or metabolic activity.

10. (canceled)

11. (canceled)

12. Method according to claim 9, wherein the control unit is connected to a remote control unit via a network, the remote control unit having user interface capability, so that input data may be transferred from the control unit and output data may be generated and displayed for a user being connected to the remote control unit via the network, preferably the remote control unit being located in a cloud-based computing environment, more preferably the remote control unit being a virtual control unit having a database, a file, a data processor for receiving input data and converting the input data to output data and displaying for a user connected to the network.

13. (canceled)

14. (canceled)

15. (canceled)

16. Method according to claim 9, wherein the control unit having user interface capability, so that input data and output data may be displayed for a user being connected to the control unit via the network.

17. Method according to claim 9, wherein the control unit is connected to a remote control unit, having user interface capability, via a network, so that input data may be transferred from the control unit and displayed for a user being connected to the remote control unit via the network.

18. Method according to claim 1, wherein the output is generated with an algorithm, machine learning or artificial intelligence.

19. Use of a method according to claim 1, for monitoring and/or controlling fiber intake.

20. A system for controlling fiber intake in mammals comprising a control unit adapted to receive input data and provide output data, at least one sample measurement device adapted to measure a sample obtained from a mammal to provide input data for the control unit, and optionally a remote control unit connected to said control unit via a network.

21. The system according to claim 20, wherein the control unit of the present system have a database, a file, and a data processor for receiving input data and converting the received input data to output data and displaying for a user connected to the network.

22. The system according to claim 20, wherein the control unit have user interface capability, so that input data and/or output data may be displayed for a user being connected to the control unit via the network.

23. The system according to claim 20, wherein the control unit is connected to a remote control unit via a network, the remote control unit having user interface capability, so that input data and/or output data may be transferred from the control unit to be displayed for a user being connected to the remote control unit via the network, preferably the remote control unit being located in a cloud-based computing environment, more preferably the remote control unit being a virtual control unit having a database, a file, and a data processor for receiving data and converting the received input data to output data and displaying for a user connected to the network.

24. (canceled)

25. (canceled)

26. (canceled)
